(19) United States
(12) Patent Application Publication
(10) Pub. No.: US 2002/0183837 A1
(43) Pub. Date: Dec. 5, 2002

(54) Apparatus and method for reducing mitral regurgitation

(76) Inventors: Richard B. Streeter, Winchester, MA (US); John R. Liddicoat, Sewickley, PA (US); William E. Cohn, Chestnut Hill, MA (US); Steven B. Woolfson, Boston, MA (US); Todd F. Davenport, Andover, MA (US)

Correspondence Address: Pandiscio & Pandiscio, 470 Totten Pond Road, Waltham, MA, 02154, US

(21) Appl. No.: 10/090,968

(22) Filed: Mar. 5, 2002

Related U.S. Application Data
(60) Provisional application No. 60/273,893, filed on Mar. 5, 2001.

(51) Int. Cl. A61F 2/24

(52) U.S. Cl. 623/2.11; 623/2.37

(57) ABSTRACT

Apparatus for reducing mitral regurgitation, by applying a force to the wall of the coronary sinus so as to force the posterior leaflet anteriorly and thereby reduce mitral regurgitation.

### REFERENCE TO RELATED APPLICATION

This patent application claims benefit of pending prior U.S. Provisional Patent Application Serial No. 60/273,893, filed Mar. 5, 2001 by William E. Cohn et al. for TRANSVASCULIR METHODS AND DEVICES FOR MITRAL VALVE PROCEDURES, which application is incorporated by reference herein.

### BACKGROUND OF THE INVENTION

Mitral valve repair is the procedure of choice to correct mitral regurgitation of all etiologies. With the use of current surgical techniques, between 70% and 95% of regurgitate mitral valves can be repaired. The advantages of mitral valve repair over mitral valve replacement are well documented. These include better preservation of cardiac function and reduced risk of anticoagulant-related hemorrhage, thromboembolism and endocarditis.

In current practice, mitral valve surgery requires an extremely invasive approach that includes a chest wall incision, cardiopulmonary bypass, cardiac and pulmonary arrest, and an incision on the heart itself to gain access to the mitral valve. Such a procedure is associated with high morbidity and mortality. Due to the risk associated with this procedure, many of the sickest patients are denied the potential benefits of surgical correction of mitral regurgitation. In addition, patients with moderate, symptomatic mitral regurgitation are denied early intervention and undergo surgical correction only after the development of cardiac dysfunction.

Mitral regurgitation is a common occurrence in patients with heart failure and a source of important morbidity and mortality in these patients. Mitral regurgitation in patients with heart failure is caused by changes in the geometric configurations of the left ventricle, papillary muscles and mitral annulus. These geometric alterations result in mitral leaflet tethering and incomplete coaptation at systole. In this situation, mitral regurgitation is corrected by plicating the mitral valve annulus, either by (i) sutures alone or by (ii) sutures in combination with a support ring, so as to reduce the circumference of the distended annulus and restore the original geometry of the mitral valve annulus.

More particularly, current surgical practice for mitral valve repair generally requires that the posterior mitral valve annulus be reduced in radius by surgically opening the left atrium and then fixing sutures, or more commonly sutures in combination with a support ring, to the internal surface of the annulus; this structure is used to cinch the annulus, in a pursestring-like fashion, to a smaller radius, thereby reducing mitral regurgitation by improving leaflet coaptation.

This method of mitral valve repair, generally termed “annuloplasty”, effectively reduces mitral regurgitation in heart failure patients. This, in turn, reduces symptoms of heart failure, improves quality of life and increases longetivity. Unfortunately, however, the invasive nature of mitral valve surgery and the attendant risks render most heart failure patients poor surgical candidates. Thus, a less invasive means to increase leaflet coaptation and thereby reduce mitral regurgitation in heart failure patients would make this therapy available to a much greater percentage of patients.

Mitral regurgitation also occurs in approximately 20% of patients suffering acute myocardial infarction. In addition, mitral regurgitation is the primary cause of cardiogenic shock in approximately 10% of patients who develop severe hemodynamic instability in the setting of acute myocardial infarction. Patients with mitral regurgitation and cardiogenic shock have about a 50% hospital mortality. Elimination of mitral regurgitation in these patients would be of significant benefit. Unfortunately, however, patients with acute mitral regurgitation complicating acute myocardial infarction are particularly high-risk surgical candidates, and are therefore not good candidates for a traditional annuloplasty procedure. Thus, a minimally invasive means to effect a temporary reduction or elimination of mitral regurgitation in these critically ill patients would afford them the time to recover from the myocardial infarction or other acute life-threatening events and make them better candidates for medical, interventional or surgical therapy.

### SUMMARY OF THE INVENTION

As a result, one object of the present invention is to provide an apparatus and method for treating mitral regurgitation which does not suffer from the disadvantages associated with conventional annuloplasty.

Another object of the present invention is to provide an apparatus and method for treating mitral regurgitation which can be deployed either permanently (e.g., for patients suffering from heart failure) or temporarily (e.g., for patients suffering from mitral regurgitation with acute myocardial infarction).

These and other objects are addressed by the present invention, which is made possible by the discovery that the mitral annulus may be remodeled without the plication of conventional, open-surgery annuloplasty.

With the above and other objects in view, a feature of the invention is the provision of an apparatus for reducing mitral regurgitation. The apparatus comprises:

a bendable elongated body adapted to be inserted into the coronary sinus of a patient in the vicinity of the posterior leaflet of the mitral valve, the elongated body being adjustable between a first configuration adapted to be delivered into the coronary sinus and a second configuration adapted to exert a force onto the posterior annulus, the body comprising:

a distal end section having a plurality of proximally-extending barbs;

a proximal end section having a plurality of distally-extending barbs; and

at least one spring segment connecting said distal end section to said proximal end section, said at least one spring segment being adapted to apply a force to said distal end section and said proximal end section so as to urge said distal end section and said proximal end section together;

whereby when said elongated body is inserted into the coronary sinus in the first configuration, said at least one spring segment will cause said elongated body to assume the second configuration so as to exert the force on the posterior annulus and thereby reduce mitral regurgitation.

In accordance with a further feature of the invention, there is provided a further apparatus for reducing mitral regurgitation. The apparatus comprises:

a variable elongated body adapted to be inserted into the coronary sinus of a patient in the vicinity of the posterior leaflet of the mitral valve, the variable elongated body being adjustable between a first configuration adapted to be delivered into the coronary sinus and a second configuration adapted to exert a force onto the posterior annulus, the variable elongated body comprising:

a first anchor comprising a first elongated section and a first anchor element disposed at one end thereof;

a second anchor having a second elongated section and a second anchor element disposed at one end thereof;

a crimp having an opening therein and being adapted to selectively close down the size of the opening;

said first anchor, said second anchor and said crimp being arranged so that said first elongated section and said second elongated section extend through said opening, with said first anchor element and said second anchor element being displaced from one another;

whereby said elongated body may be positioned in said first configuration wherein first anchor element and said second anchor element are displaced from one another by a first distance, said elongated body may be deployed in said coronary sinus, and said elongated body may thereafter be moved into said second configuration wherein said first anchor and said second anchor are displaced from one another by a second, shorter distance, whereby to exert the force on the posterior annulus and thereby reduce mitral is regurgitation.

In accordance with a further feature of the invention, there is provided a method for reducing mitral regurgitation. The method comprises the steps of:

providing a prosthesis comprising:

a bendable elongated body adapted to be inserted into the coronary sinus of a patient in the vicinity of the posterior leaflet of the mitral valve, the elongated body being adjustable between a first configuration adapted to be delivered into the coronary sinus and a second configuration adapted to exert a force onto the posterior annulus, the body comprising:

a distal end section having a plurality of proximally-extending barbs;

a proximal end section having a plurality of distally-extending barbs; and

at least one spring segment connecting said distal end section to said proximal end section, said at least one spring segment being adapted to apply a force to said distal end section and said proximal end section so as to urge said distal end section and said proximal end section together;

whereby when said elongated body is inserted into the coronary sinus in the first configuration, said at least one spring segment will cause said elongated body to assume the second configuration so as to exert the force on the posterior annulus and thereby reduce mitral regurgitation;

positioning the prosthesis in the coronary sinus while in the first configuration; and

reconfiguring the prosthesis into the second configuration.

In accordance with a further feature of the invention, there is provided a further method for reducing mitral regurgitation, the method comprising the steps of:

providing a prosthesis comprising:

a variable elongated body adapted to be inserted into the coronary sinus of a patient in the vicinity of the posterior leaflet of the mitral valve, the variable elongated body being adjustable between a first configuration adapted to be delivered into the coronary sinus and a second configuration adapted to exert a force onto the posterior annulus, the variable elongated body comprising:

a first anchor comprising a first elongated section and a first anchor element disposed at one end thereof;

a second anchor having a second elongated section and a second anchor element disposed at one end thereof;

a crimp having an opening therein and being adapted to selectively close down the size of the opening;

said first anchor, said second anchor and said crimp being arranged so that said first elongated section and said second elongated section extend through said opening, with said first anchor element and said second anchor element being displaced from one another;

whereby said elongated body may be positioned in said first configuration wherein first anchor element and said second anchor element are displaced from one another by a first distance, said elongated body may be deployed in said coronary sinus, and said elongated body may thereafter be moved into said second configuration wherein said first anchor and said second anchor are displaced from one another by a second, shorter distance, whereby to exert the force on the posterior annulus and thereby reduce mitral regurgitation;

positioning the prosthesis in the coronary sinus while in the first configuration; and reconfiguring the prosthesis into the second configuration.

The above and other features of the invention, including various novel details of construction and combinations of parts and method steps, will now be more particularly described with reference to the accompanying drawings and pointed out in the claims. It will be understood that the particular devices and methods embodying the invention are shown by way of illustration only and not as limitations of the invention. The principles and features of this invention may be employed in various and numerous embodiments without departing from the scope of the invention.

### BRIEF DESCRIPTION OF THE DRAWINGS

The above and other objects and features of the present invention are more fully disclosed by the following detailed description of the preferred embodiments of the invention, which is to be considered together with the accompanying drawings wherein like numbers refer to like parts and further wherein:

FIG. 1 is a schematic view of portions of the human vascular system;

FIG. 2 is a schematic view of portions of the human heart;

FIG. 3 is a side elevational, partly sectional view of a preferred apparatus formed in accordance with the present invention and shown in a first configuration;

FIGS. 4 is a sectional view taken along line IV-IV of FIG. 3;

FIG. 5 is a side elevational view of the apparatus of FIG. 3 shown in a second configuration;

FIG. 6 is a diagrammatic illustration of an alternative embodiment in a first configuration;

FIG. 7 is a diagrammatic illustration of the embodiment of FIG. 6 in a second configuration;

FIG. 8 is a diagrammatic illustration of another alternative embodiment;

FIG. 9 is similar to FIG. 8, but illustrative of the embodiment of FIG. 8 in a second configuration;

FIG. 10 is a schematic view of portions of the human heart and illustrating diagrammatically another alternative embodiment of the invention;

FIG. 11 is a diagrammatic illustration of another alternative embodiment of the present invention;

FIG. 12 is a diagrammatic illustration of still another alternative embodiment of the present invention, with the embodiment being shown in a first configuration and a second configuration;

FIGS. 13 and 14 show the embodiment of FIG. 12 applied to the anatomy of a patient, with FIG. 13 showing the embodiment in the aforementioned first configuration and FIG. 14 showing the embodiment in the aforementioned second configuration; and

FIGS. 15A-15E are a series of diagrammatic illustrations showing deployment of the embodiment of FIGS. 12-14.

### DETAILED DESCRIPTION OF THE PREFERRED EMBODIMENTS

The coronary sinus is the largest vein in the human heart. During a large portion of its course in the atrioventricular groove, the coronary sinus typically extends adjacent to the left atrium of the heart for a distance of approximately 5 to 10 centimeters. Significantly, for a portion of its length, e.g., typically approximately 7-9 cm, the coronary sinus extends substantially adjacent to the posterior perimeter of the mitral annulus. The present invention takes advantage of this fact. More particularly, by deploying an elongated body in the coronary sinus, adjacent to the posterior leaflet of the mitral valve, pressure may be brought to bear on the posterior annulus of the mitral valve, whereby to move the posterior annulus anteriorly so as to improve leaflet coaptation and, as a result, reduce mitral regurgitation. In this respect it should be appreciated that the posterior annulus may be shifted anteriorly so as to achieve, or to attempt to achieve to the extent anatomically possible, leaflet-to-leaflet engagement or leaflet-to-annulus engagement (e.g., where a leaflet may be tethered due to left ventricular distortion). Both of these types of engagement, or targeted engagement, are intended to be encompassed by the terms “improved leaflet coaptation” and/or “increased leaflet coaptation” and the like.

In one preferred embodiment of the invention, access to the coronary sinus is gained percutaneously, e.g., the elongated body is introduced into the patient's vascular system via the jugular vein or via the left subclavian vein, passed down the superior vena cava, passed through the right atrium and then passed into the coronary sinus, where it is deployed. Alternatively, the elongated body may be introduced into the coronary sinus through a small incision in the heart, or through some other incision into the patient's vascular system.

Once deployed, the elongated body may be left in position permanently (e.g., in the case of patients suffering from mitral regurgitation associated with heart failure) or the elongated body may be left in position only temporarily (e.g., in the case of patients suffering from mitral regurgitation associated with acute myocardial infarction).

Visualization of the procedure may be obtained by fluoroscopy, echocardiography, intravascular ultrasound, angioscopy, real-time magnetic resonance imaging, etc. The efficacy of the procedure may be determined through echocardiography, although other imaging modalities may also be suitable.

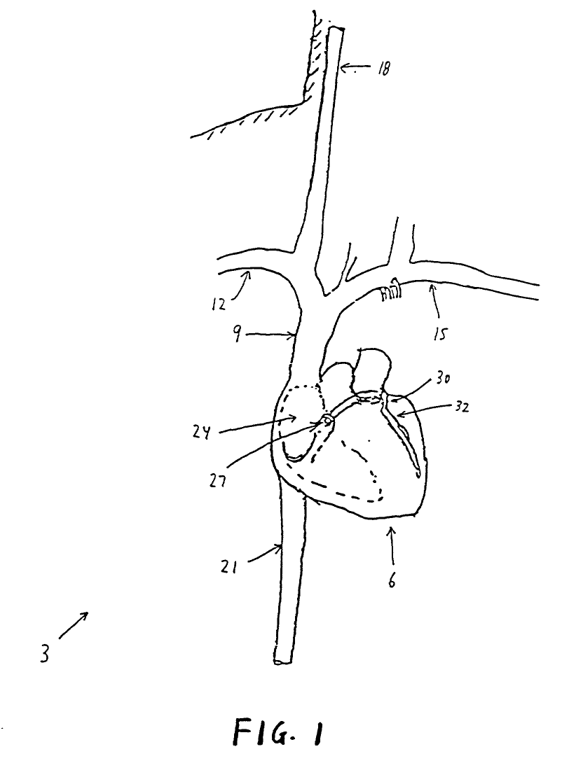

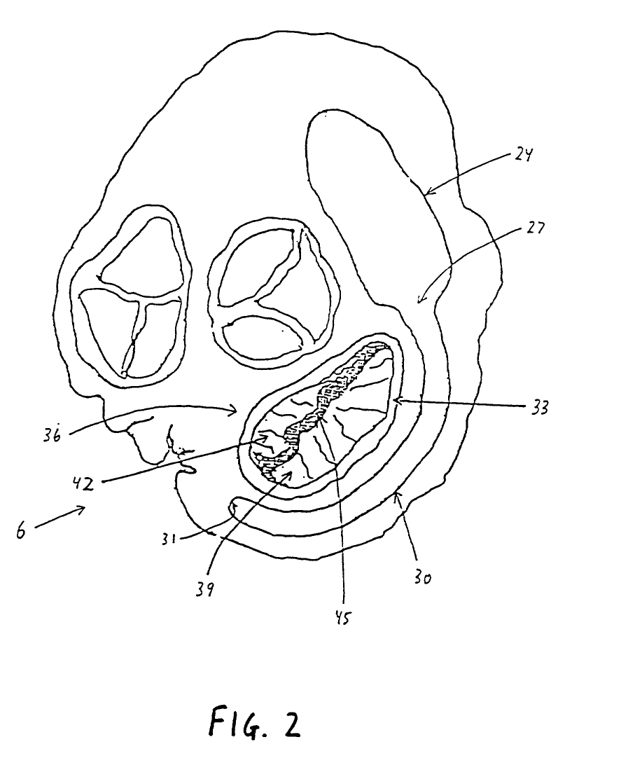

Looking now at FIG. 1, there are shown aspects of the cardiovascular system 3 of a patient. More particularly, cardiovascular system 3 generally comprises the heart 6, the superior vena cava 9, the right subclavian vein 12, the left subclavian vein 15, the jugular vein 18, and the inferior vena cava 21. Superior vena cava 9 and inferior vena cava 21 communicate with the heart's right atrium 24. The coronary ostium 27 leads to coronary sinus 30. At the far end 31 (FIG. 2) of coronary sinus 30, the vascular structure turns into she vertically-descending anterior interventricular vein (“AIV”) 32 (see FIG. 1). For purposes of the present invention, it can generally be convenient to consider the term “coronary sinus” to mean the vascular structure extending between coronary ostium 27 and AIV 32.

As seen in FIG. 2, between coronary ostium 27 and AIV 32, coronary sinus 30 generally extends substantially adjacent to the posterior perimeter of the annulus 33 of the mitral valve 36. Mitral valve 36 comprises a posterior leaflet 39 and an anterior leaflet 42. In the case of a regurgitant mitral valve, posterior leaflet 39 and anterior leaflet 42 will generally fail to properly coapt at systole, thereby leaving an intervening gap 45 which will permit regurgitation.

Referring to FIG. 3, it will be seen that an illustrative preferred embodiment includes an elongated flexible body 50. The body 50 preferably is provided with a rounded or pointed distal end 52 for insertion into the coronary sinus 30 (FIG. 5).

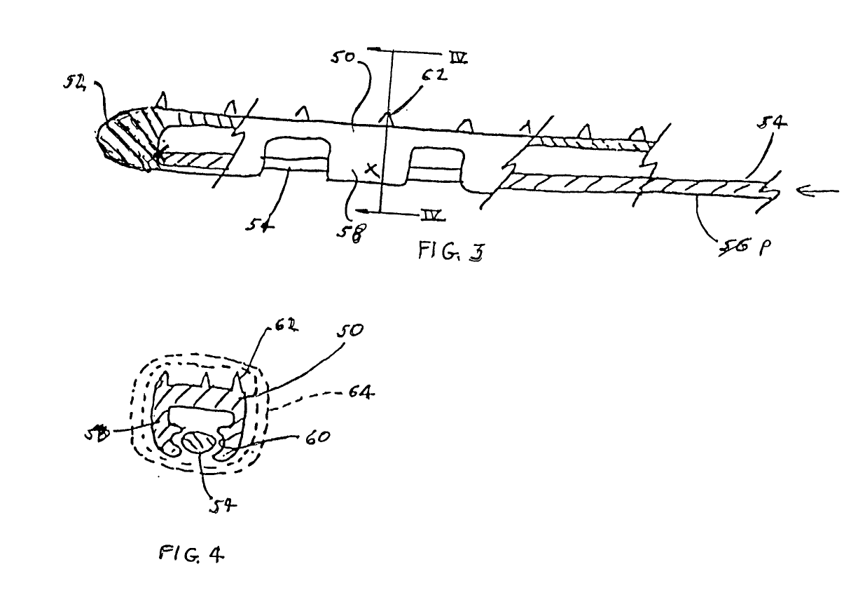

Fixed to the distal end 52 of the body 50 is a wire 54 which extends through the body 50, with a proximal portion P thereof extending proximally from body 50 (FIG. 3). The body 50 is provided with wire supporting portions 58, each of which defines a channel 60 (FIG. 4) for retaining the wire 54, but permitting the wire 54 to slide therethrough. Wire 54 is preferably positioned on one side of the longitudinal axis of body 50, and body 50 preferably includes a plurality of openings 55 helping to define a plurality of flexible bridges 56.

The body 50 may be provided with barbs 62 for engagement with tissue in the coronary sinus 30. When barbs 62 are used, the elongated body 50 should be housed in a guide catheter 64 (FIG. 4) which is removed once the body 50 is in place, to expose barbs 62.

As body 50 is inserted into coronary sinus 30, it will generally assume the shape of the coronary sinus, which is naturally curved in the region of the posterior leaflet of the mitral valve. Thereafter, wire 54 may be pushed or pulled, as desired, so as to alter the configuration of body 50. More specifically, by pushing the wire 54 in a distal direction, the body 50 is caused to reconfigure to a tighter arc around the mitral valve annulus 33, i.e., by bending on bridges 56 and enlarging openings 55. By pulling the wire 54 proximally, the body is caused to reconfigure to a more extended arc, or to assume a straight configuration, or even to assume an inverted configuration, by bending on bridges 56 and reducing openings 55. Either alteration of the configuration of body 50 in turn alters the configuration of the coronary sinus adjacent to the mitral valve, whereby to force the posterior annulus anteriorly and thereby improve leaflet coaptation and hence reduce mitral regurgitation.

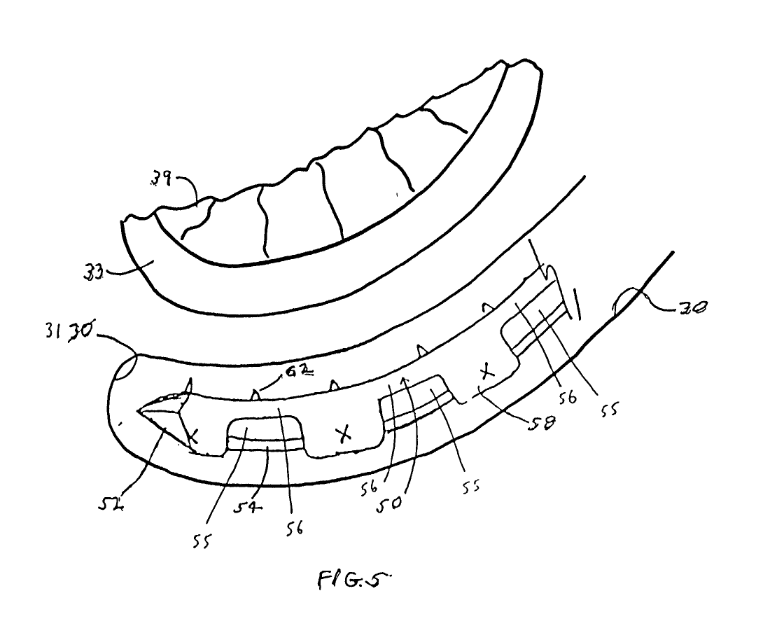

Looking next at FIG. 6, there is shown an alternative embodiment of the present invention. More particularly, there is shown an elongated body 100 which comprises a plurality of staples 103 connected by a flexible bridge 105. A wire 110 has one end secured to the distalmost end of bridge 105. During use, the elongated body 100 is positioned within the coronary sinus (FIG. 7), staples 103 are secured to the walls of the coronary sinus 30, and then wire 110 is pushed distally or pulled proximally so as to modify the configuration of elongated body 100. More particularly, pulling wire 110 proximally will cause bridge 105 to reconfigure to a tighter arc around the mitral valve annulus, whereas pushing wire 110 distally will cause bridge 105 to reconfigure into a more extended arc, or to go straight, or even to invert. This action in turn alters the configuration of the coronary sinus 30 adjacent to the mitral valve 36, whereby to force the posterior annulus anteriorly and thereby improve leaflet coaptation and hence reduce mitral regurgitation.

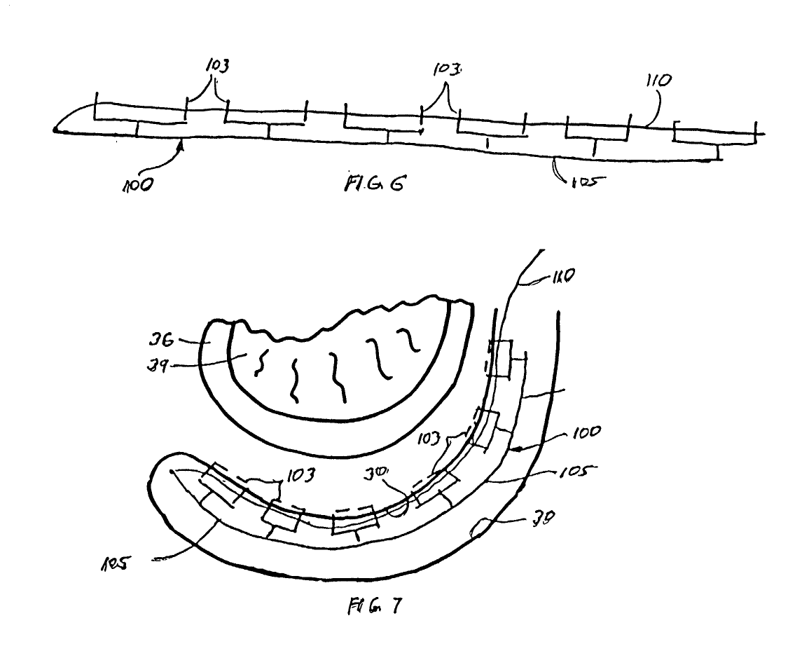

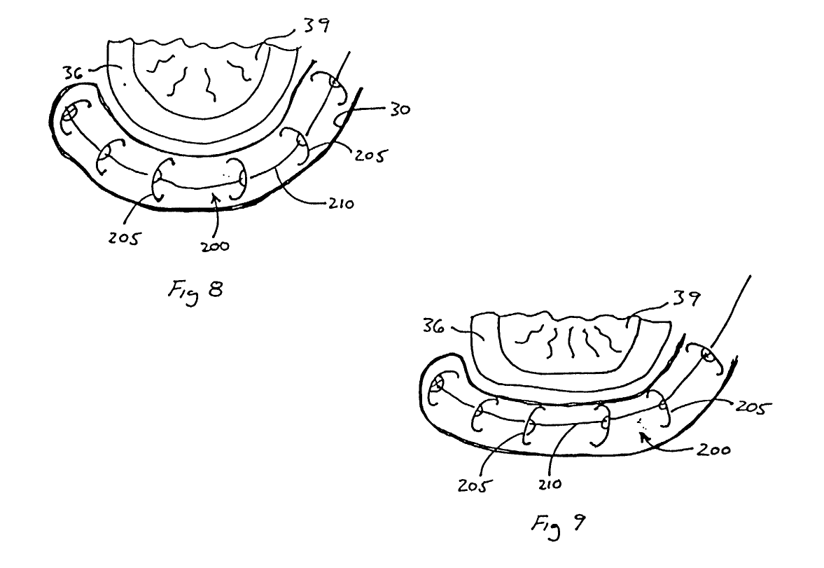

Looking next at FIG. 8, there is shown another alternative embodiment of the present invention. More particularly, there is shown an elongated body 200 which comprises a plurality of anchors 205, formed by staples, or the like, each comprising an eyelet through which extends a wire 210. The distal end of wire 210 is secured to the distalmost staple. During use, the elongated body 200 is positioned within the coronary sinus, the anchors 203 are secured to the walls of the coronary sinus 30, and then wire 210 is pulled proximally so as to modify the configuration of elongated body 200. More specifically, pulling of the wire 210 causes the body 200 to reconfigure to a wider arc (FIG. 9) and then, if pulled further, to a substantially straight configuration. Such action, in turn, alters the configuration of the coronary sinus 30 adjacent to the mitral valve 36, whereby to force the posterior annulus anteriorly and thereby improve leaflet coaptation and hence reduce mitral regurgitation.

Looking next at FIG. 10, there is shown another embodiment of the present invention. More particularly, there is shown an elongated body 300 which is adapted to reducing mitral regurgitation by scarring the mitral valve annulus 33 to cause contraction thereof. Elongated body 300 includes an element at its distal end which is adapted to inject a scarring medium into the mitral valve annulus. This scarring medium may comprise a chemical, or it may comprise energy selected from a group of energies consisting of thermal, cryogenic, laser and radio frequency.

Looking next at FIG. 11, there is shown an elongated body 400 which comprises a self-cinching version of the invention. More particularly, body 400 comprises a distal end section 405 including a plurality of proximally-extending barbs 410, a proximal end section 415 including a plurality of distally-extending barbs 420, and one or more spring segments 425 connecting distal end section 405 to proximal end section 415. If desired, intermediate sections 430, with or without associated barbs 435, may be disposed between spring segments 425.

In use, elongated body 400 is positioned in coronary sinus 30 with its one or more spring sections 405 configured in an extended condition, and then the one or more spring sections 425 are reconfigured into a contracted condition so that the device's distal end section 405 and proximal end section 415 are drawn together. This action will cause barbs 410 and 420 to set into the surrounding tissue and draw this tissue closer together. With elongated body 400 residing in coronary sinus 30 and drawing separated sections of the curved coronary sinus closer together, the coronary sinus is effectively straightened and the posterior leaflet 39 is forced anteriorly, whereby to reduce or completely eliminate mitral regurgitation.

If desired, the one or more spring sections 425 may be formed out of a resilient material, e.g., a resilient metal or plastic. In this case, the one or more spring sections 425 may be restrained in an extended condition when the elongated body 400 is positioned in the coronary tissue; and the one or more spring sections 425 may thereafter be released so as to draw together distal end section 405 and proximal end section 415. Alternatively, the one or more spring sections 425 may be formed out of a so-called shape memory alloy, with a temperature transition being used to effect the desired shortening of the one or more spring sections 425 when the coronary sinus is to be straightened.

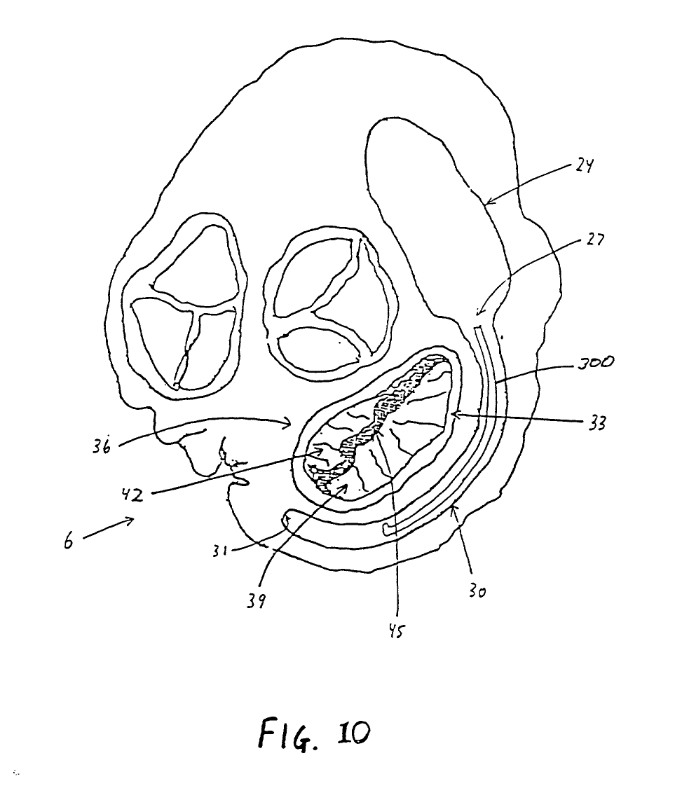

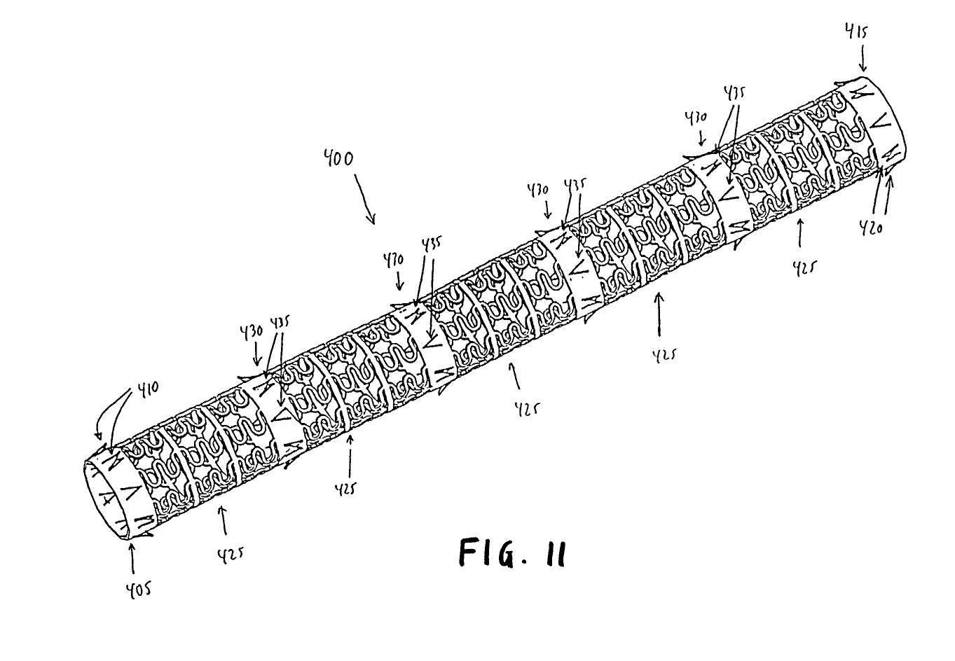

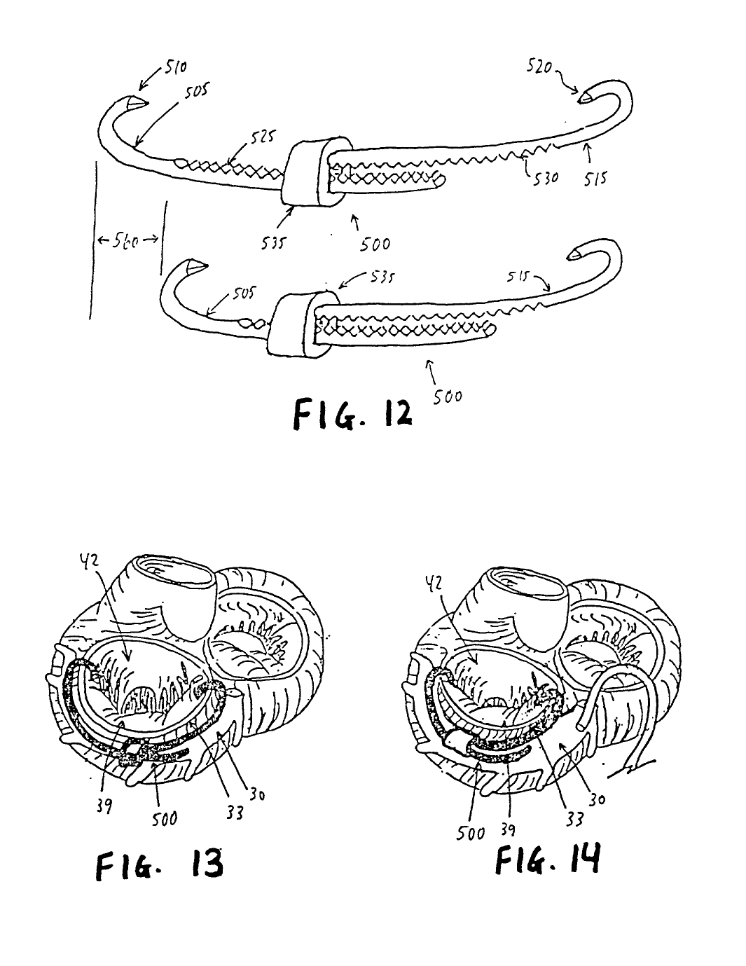

Looking next at FIG. 12, there is shown a device 500 which is intended to minimize trauma to the coronary sinus wall by fixating at only two points and then cinching between those two points, using the coronary sinus as a guide path for the cinching mechanism. To this end, device 500 comprises an anterolateral anchor 505 having a hook 510 and a posteromedial anchor 515 having a hook 520. Hooks 510 and 520 are shown in FIG. 12 as simple curved shapes with sharp tips, however, other configurations may also be used, e.g., barbs or staples or suture knots. A ratcheting mechanism is preferably used to effect cinching between anchors 505 and 515. In one preferred embodiment of the present invention, the ratcheting mechanism is bi-directional and is achieved by creating a rough or saw-toothed surface 525 on anchor 505 and a rough or saw-toothed surface 530 on anchor 515. Fixation crimp 535 forces the two surfaces 525 and 530 together so as to keep them from slipping relative to one another. In one form of the invention, fixation crimp 535 is an elastomeric material of sufficient durometer to allow the two anchors to be forced apart when desired but which will normally not move under the loads associated with cardiac function. Alternatively, crimp 535 may be formed of a deformable material that is crimped after the two anchors 505 and 510 have been cinched together.

In use, device 500 is deployed in the coronary sinus 30 with its anchors 505 and 515 in an extended configuration; hooks 510 and 520 are set into the wall of the coronary sinus; and then anchors 505 and 515 are ratched together so as to bring hooks 510 and 520 (and hence remote portions of tissue) together, whereby to straighten the coronary sinus and thereby reduce mitral regurgitation. FIG. 13 shows the device 500 in an initial, uncinched configuration, and FIG. 14 shows the device 500 in a final, cinched configuration, with the resulting plication of mitral annulus 33.

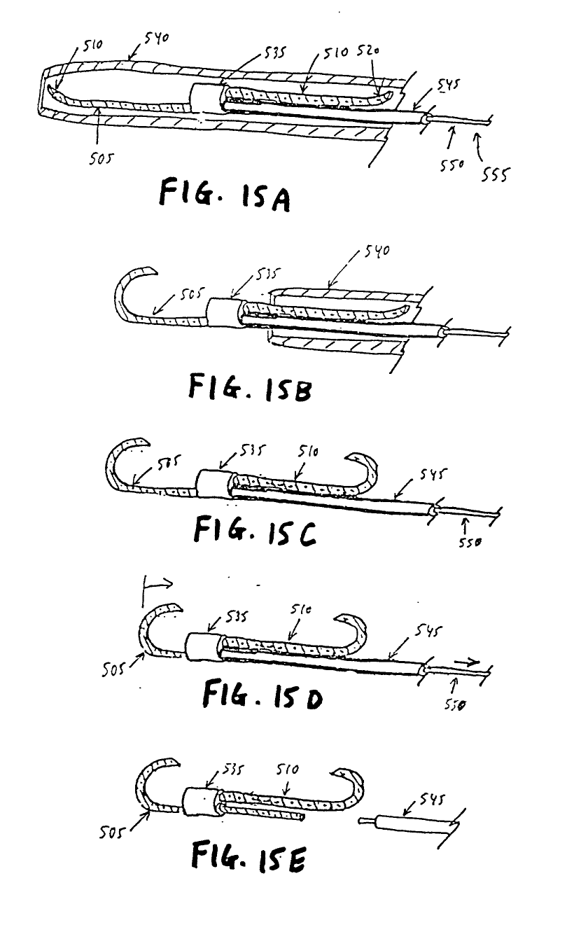

FIGS. 15A-15E show one embodiment of device 500 being deployed and cinched. Device 500 is introduced into the coronary sinus via an outer sheath 540. Hooks 510 and 520 are made of a resilient material such as Nitinol, stainless steel or plastic, and are stretched flat by the outer sheath 540. A push rod 545 and pull rod 550 form a cinching device 555. In this embodiment, push rod 545 is temporarily connected to device 500 by a threaded hole in crimp 535. Pull rod is temporarily connected to device 500 by a threaded hole in the proximal end of anterolateral anchor 505. When push rod 545 and pull rod 550 are moved in relationship to each other, the device 500 changes length by a distance 560 shown in FIG. 12.

FIG. 15B shows anchor 505 emerging from outer sheath 540 and regaining its original curved shape. This can be effected by pushing the device 500 distally with push rod 545. As anchor 505 emerges from outer sheath 540, its hook 510 will engage in the adjacent tissue.

FIG. 15C shows anchor 515 emerging from sheath 540 and regaining its original curved shape. As anchor 515 emerges from outer sheath 540, its hook 520 will engage in the adjacent tissue.

FIG. 15D shows pull rod 550 being moved relative to push rod 545 so as to reduce the overall length of device 500. As this occurs, opposing hooks 510 and 520 will draw the tissue together, so as to plicate mitral annulus 33 and thereby reduce mitral regurgitation.

FIG. 15E shows cinching device 555 removed from annuloplasty device 500 by unscrewing both pull rod 555 and push rod 545.

It is to be understood that the present invention is by no means limited to the particular constructions and method steps herein disclosed and/or shown in the drawings, but also comprises any modifications or equivalents within the scope of the claims.

What is claimed is:

1. Apparatus for reducing mitral regurgitation, the apparatus comprising:
a bendable elongated body adapted to be inserted into the coronary sinus of a patient in the vicinity of the posterior leaflet of the mitral valve, the elongated body being adjustable between a first configuration adapted to be delivered into the coronary sinus and a second configuration adapted to exert a force onto the posterior annulus, the body comprising:
a distal end section having a plurality of proximally-extending barbs;
a proximal end section having a plurality of distally-extending barbs; and
at least one spring segment connecting said distal end section to said proximal end section, said at least one spring segment being adapted to apply a force to said distal end section and said proximal end section so as to urge said distal end section and said proximal end section together;
whereby when said elongated body is inserted into the coronary sinus in the first configuration, said at least one spring segment will cause said elongated body to assume the second configuration so as to exert the force on the posterior annulus and thereby reduce mitral regurgitation.

2. Apparatus according to claim 1 wherein the at least one spring segment comprises an elastic material, and further wherein said elastic material is stretched when said elongated body is in the first configuration and said elastic material is relaxed when said elongated body is in the second configuration.

3. Apparatus according to claim 1 wherein said at least one spring segment comprises a shape memory alloy, and further wherein a temperature transition is used to transform said elongated body from the first configuration to the second configuration.

4. Apparatus according to claim 1 wherein there are at least two spring segments connecting said distal end section to said proximal end section, and further wherein an intermediate section is disposed between said at least two spring segments.

5. Apparatus according to claim 4 wherein said intermediate section has at least one barb thereon.

6. Apparatus for reducing mitral regurgitation, the apparatus comprising:
a variable elongated body adapted to be inserted into the coronary sinus of a patient in the vicinity of the posterior leaflet of the mitral valve, the variable elongated body being adjustable between a first configuration adapted to be delivered into the coronary sinus and a second configuration adapted to exert a force onto the posterior annulus, the variable elongated body comprising:
a first anchor comprising a first elongated section and a first anchor element disposed at one end thereof;
a second anchor having a second elongated section and a second anchor element disposed at one end thereof;
a crimp having an opening therein and being adapted to selectively close down the size of the opening;
said first anchor, said second anchor and said crimp being arranged so that said first elongated section and said second elongated section extend through said opening, with said first anchor element and said second anchor element being displaced from one another;
whereby said elongated body may be positioned in said first configuration wherein first anchor element and said second anchor element are displaced from one another by a first distance, said elongated body may be deployed in said coronary sinus, and said elongated body may thereafter be moved into said second configuration wherein said first anchor and said second anchor are displaced from one another by a second, shorter distance, whereby to exert the force on the posterior annulus and thereby reduce mitral regurgitation.

7. Apparatus according to claim 6 wherein said first anchor element comprises a first hook at one end of said first elongated section.

8. Apparatus according to claim 6 wherein said second anchor element comprises a second hook at one end of said first elongated section.

9. Apparatus according to claim 6 wherein said crimp is made of elastic material.

10. Apparatus according to claim 9 wherein at least one of said anchors comprises a saw-toothed surface for engaging the other of said anchors.

11. Apparatus according to claim 6 wherein said crimp is made of a material which will take a set.

12. A method for reducing mitral regurgitation, the method comprising the steps of:
providing a prosthesis comprising:
a bendable elongated body adapted to be inserted into the coronary sinus of a patient in the vicinity of the posterior leaflet of the mitral valve, the elongated body being adjustable between a first configuration adapted to be delivered into the coronary sinus and a second configuration adapted to exert a force onto the posterior annulus, the body comprising:
a distal end section having a plurality of proximally-extending barbs;
a proximal end section having a plurality of distally-extending barbs; and
at least one spring segment connecting said distal end section to said proximal end section, said at least one spring segment being adapted to apply a force to said distal end section and said proximal end section so as to urge said distal end section and said proximal end section together;
whereby when said elongated body is inserted into the coronary sinus in the first configuration, said at least one spring segment will cause said elongated body to assume the second configuration so as to exert the force on the posterior annulus and thereby reduce mitral regurgitation;
positioning the prosthesis in the coronary sinus while in the first configuration; and
reconfiguring the prosthesis into the second configuration.

13. A method for reducing mitral regurgitation, the method comprising the steps of:
providing a prosthesis comprising:
a variable elongated body adapted to be inserted into the coronary sinus of a patient in the vicinity of the posterior leaflet of the mitral valve, the variable elongated body being adjustable between a first configuration adapted to be delivered into the coronary sinus and a second configuration adapted to exert a force onto the posterior annulus, the variable elongated body comprising:
a first anchor comprising a first elongated section and a first anchor element disposed at one end thereof;
a second anchor having a second elongated section and a second anchor element disposed at one end thereof;
a crimp having an opening therein and being adapted to selectively close down the size of the opening;
said first anchor, said second anchor and said crimp being arranged so that said first elongated section and said second elongated section extend through said opening, with said first anchor element and said second anchor element being displaced from one another;
whereby said elongated body may be positioned in said first configuration wherein first anchor element and said second anchor element are displaced from one another by a first distance, said elongated body may be deployed in said coronary sinus, and said elongated body may thereafter be moved into said second configuration wherein said first anchor and said second anchor are displaced from one another by a second, shorter distance, whereby to exert the force on the posterior annulus and thereby reduce mitral regurgitation;
positioning the prosthesis in the coronary sinus while in the first configuration; and
reconfiguring the prosthesis into the second configuration.
